(19) United States
(12) Patent Application Publication
(10) Pub. No.: US 2023/0346843 A1
(43) Pub. Date: Nov. 2, 2023

(54) Stem Cells for Treatment of Respiratory Disorders

(71) Applicant: Xintela AB, Lund (SE)

(72) Inventors: Evy Lundgren Åkerlund, Bjärred (SE); Sandra Lindstedt, Lund (SE); Christina Uvebrant, Malmö (SE); Tania Ramos Moreno, Lund (SE)

(73) Assignee: Xintela AB, Lund (SE)

(21) Appl. No.: 17/923,124

(22) PCT Filed: May 7, 2021
(86) PCT No.: PCT/EP2021/062111

§ 371 Date: Nov. 3, 2022

(30) Foreign Application Priority Data
May 7, 2020 (EP) 20173592.5

(51) Int. Cl. A61K 35/28; C12N 5/0775; A61P 37/06; A61P 11/00

(52) U.S. Cl. CPC A61K 35/28; C12N 5/0668; A61P 37/06; A61P 11/00

(57) ABSTRACT

The present invention concerns the use of integrin alpha10-selected mesenchymal stem cells for treatment of respiratory tract disorders, diseases and trauma affecting the respiratory tract.

### TECHNICAL FIELD

The present invention relates to integrin alpha10-selected mesenchymal stem cells, and their use in the treatment of respiratory disorders.

### BACKGROUND

There is a large unmet clinical need to treat respiratory tract disorders, such as acute distress syndrome (ARDS), a severe type of respiratory failure characterized by rapid onset of widespread inflammation in the lung. ARDS has a significant impact on critical care patients and still remains as a devastating clinical disorder associated with high mortality rates (estimated to be around 40%), where those who survive can experience significant long-term morbidity.

Symptoms include shortness of breath, rapid breathing, and bluish skin coloration. There are direct and indirect causes of ARDS depending whether the lungs are initially affected. Direct causes include pneumonia (including bacterial or viral infection, for example caused by COVID-19), aspiration, inhalational lung injury, lung contusion, chest trauma, and near-drowning. Indirect causes include sepsis, shock, pancreatitis, trauma, cardiopulmonary bypass or burns (Matthay 2019). Fewer cases of ARDS are linked to large volumes of fluid used during post-trauma resuscitation (Casay 2019).

ARDS is typically provoked by an acute injury that results in a massive immunological response and the release of a multitude of immunological mediators, a phenomenon referred to as cytokine storm, which can affect several organs, for example the lungs (Gonzales 2015). Since ARDS is characterized by an extensive activation of the immune system, thereby causing massive damage to lung tissue, specific drugs targeting certain molecules or pathways are of limited effect. Recent advances in the management of ARDS have mostly been achieved in supportive care, and aimed to protect the respiratory exchange, preserving life and allowing physicians to wait for the resolution of the underlying disease (Prescott 2016). These strategies include the use of protective mechanical ventilation, neuromuscular blocking agents, prone positioning, and conservative fluid strategies. The latter, however, does not represent the real treatment of ARDS since it is aimed to preserve the respiratory exchange. To further reduce mortality, the therapy of ARDS should target the inflammatory mechanisms responsible of the lung injury. However, to date, no pharmacologic therapy has been able to act effectively on disease-specific pathways or to reduce mortality. Accordingly, there is a need for novel treatments for ARDS and associated disorders.

### SUMMARY

The present inventors have found that integrin alpha10-selected mesenchymal stem cells (MSCs) improve hemodynamic stability and oxygenation capacity as well as reduce blod clot formation and lung tissue damage in an animal model for ARDS. In addition the MSCs show specific immunomodulatory and anti-inflammatory propertis. This makes integrin alpha10-selected MSCs particularly suitable to alleviate the effects of respiratory disorders such as ARDS and associated lung complications.

In a main aspect, the present invention concerns a composition comprising integrin alpha10-selected Mesenchymal Stem Cells (MSC), for use in the treatment of one or more disease(s) or trauma(s) of the respiratory system/tract; and/or in connection with transplantation of one or more organs or tissue of the respiratory tract of a mammal.

In a further aspect, the present disclosure is directed to a method of treatment of a disease, disorder or trauma of the respiratory system of a mammal, the method comprising administering a therapeutically effective amount of a composition comprising integrin α10-selected Mesenchymal Stem Cells (MSCs).

In a further aspect, the present disclosure is directed to a method of treatment or promotion of a transplantation of an organ or tissue of the respiratory tract of a mammal, the method comprising administering a therapeutically effective amount of a composition comprising integrin α10-selected Mesenchymal Stem Cells (MSCs).

In a further aspect, the present disclosure is directed to a method of preventing blood clotting in a mammal in connection with a disease, disorder, or trauma of the respiratory system, and/or in connection with transplantation of an organ or tissue of the respiratory tract of a mammal, the method comprising administering a therapeutically effective amount of a composition comprising integrin α10-selected Mesenchymal Stem Cells (MSCs).

In a further aspect, the present disclosure is directed to a method of promoting hemodynamic stability in connection with a disease, disorder, or trauma of the respiratory system, and/or in connection with transplantation of an organ or tissue of the respiratory tract of a mammal, the method comprising administering a therapeutically effective amount of a composition comprising integrin α10-selected Mesenchymal Stem Cells (MSCs).

In a further aspect, the present disclosure is directed to a method of reducing the need for inotropic support in connection with a disease, disorder, or trauma of the respiratory system, and/or in connection with transplantation of an organ or tissue of the respiratory tract of a mammal, the method comprising administering a therapeutically effective amount of a composition comprising integrin α10-selected Mesenchymal Stem Cells (MSCs).

In a further aspect, the present disclosure is directed to a method of improving oxygenation capacity in connection with a disease, disorder, or trauma of the respiratory system, and/or in connection with transplantation of an organ or tissue of the respiratory tract of a mammal, the method comprising administering a therapeutically effective amount of a composition comprising integrin α10-selected Mesenchymal Stem Cells (MSCs).

In a further aspect, the present disclosure is directed to a method of preventing tissue damage, for example structural tissue damage, in connection with a disease, disorder, or trauma of the respiratory system, and/or in connection with transplantation of an organ or tissue of the respiratory tract of a mammal, the method comprising administering a therapeutically effective amount of a composition comprising integrin α10-selected Mesenchymal Stem Cells (MSCs).

In a further aspect, the present disclosure is directed to a method of reverting tissue damage, for example structural tissue damage, in connection with a disease, disorder, or trauma of the respiratory system, and/or in connection with transplantation of an organ or tissue of the respiratory tract of a mammal, the method comprising administering a therapeutically effective amount of a composition comprising integrin α10-selected Mesenchymal Stem Cells (MSCs).

In a further aspect, the present disclosure is directed to a method of reducing neutrophil counts in connection with a disease, disorder, or trauma in connection with a disease, disorder, or trauma of the respiratory system, and/or in connection with transplantation of an organ or tissue of the respiratory tract of a mammal, the method comprising administering a therapeutically effective amount of a composition comprising integrin α10-selected Mesenchymal Stem Cells (MSCs).

In a further aspect, the present disclosure is directed to a method of increasing lymphocyte counts in connection with a disease, disorder, or trauma in connection with a disease, disorder, or trauma of the respiratory system, and/or in connection with transplantation of an organ or tissue of the respiratory tract of a mammal, the method comprising administering a therapeutically effective amount of a composition comprising integrin α10-selected Mesenchymal Stem Cells (MSCs).

In a further aspect, the present disclosure is directed to a method of decreasing proinflammatory cytokines in connection with a disease, disorder, or trauma in connection with a disease, disorder, or trauma of the respiratory system, and/or in connection with transplantation of an organ or tissue of the respiratory tract of a mammal, the method comprising administering a therapeutically effective amount of a composition comprising integrin α10-selected Mesenchymal Stem Cells (MSCs).

In a further aspect, the present disclosure is directed to a method of increasing interferon-α in connection with a disease, disorder, or trauma in connection with a disease, disorder, or trauma of the respiratory system, and/or in connection with transplantation of an organ or tissue of the respiratory tract of a mammal, the method comprising administering a therapeutically effective amount of a composition comprising integrin α10-selected Mesenchymal Stem Cells (MSCs).

In a further aspect, the present disclosure is directed to a composition comprising integrin alpha10-selected Mesenchymal Stem Cells (MSCs), for use in a method of
preventing blood clotting,
promoting hemodynamic stability,
reducing the need for inotropic support,
improving oxygenation capacity,
preventing tissue damage, for example structural tissue damage,
reverting tissue damage, for example structural tissue damage,
reducing neutrophil counts,
increasing lymphocyte counts,
decreasing proinflammatory cytokines, and/or
increasing interferon-α,
in a mammal in connection with a disease, disorder, or trauma of the respiratory system, and/or in connection with transplantation of an organ or tissue of the respiratory tract of a mammal.

### DESCRIPTION OF DRAWINGS

FIG. 1: Less Inotropic Support Needed in MSC-Treated Animals

ARDS was induced in 12 pigs and the pigs were treated either with intravenous injections of integrin alpha10-selected MSCs (6 pigs) or with the cryopreservation solution with DMSO (6 pigs). Inotropic support such as administration of norepinephrine is given for ensuring hemodynamic stability and oxygenation levels in the ARDS model during the course of experiment. Thus, the amount of norepinephrine that is administered is a way to determine the hemodynamic stability in the animals and the oxygenation capacity of the lungs. We found that the total amount of administrated norepinephrine was significantly less in the MSC-treated animals (Treatment) compared to the control group (Non-Treatment). This indicates that animals treated with the integrin alpha10-selected MSCs have a more stable hemodynamic and can ensure a better oxygen distribution. **** indicates p≤0.001

FIG. 2: Improved Oxygenation Capacity with MSC Treatment.

Twelve hours after treatment started, we could analyse oxygenation capacity in three of the MSC-treated pigs and three of the control pigs. Oxygen capacity was measured by analysing the ratio between the arterial oxygen partial pressure (PaO₂) and inspired oxygen (FIO₂). The results show that the MSC-treated pigs (Treatment) showed improved oxygenation capacity (PaO₂/FIO₂ ratio: 21-28) compared to the control pigs (Non-Treatment) (PaO₂/FIO₂ ratio: X-X). This indicates a better-preserved lung structure for gas exchange in the MSC-treated animals compared to the control animals.

FIG. 3: Increased Coagulation Time in MSC-Treated Animals.

We investigated the coagulation (blood clotting) time in the ARDS model since coagulation is a predominant characteristic of ARDS, leading to extreme inflammatory response. The coagulation time was monitored in both MSC-treated (circles) and non-treated (squared) groups. The graph displays the clotting time (in seconds, s) at the different stages of the experiment and shows an increased clotting time in the MSC-treated group (circles). The effect in the treated group can be appreciated already 2 h after the infusion of integrin alpha10-selected MSCs, and lasts for at least 12 h. Increased clotting times relate to a less ARDS and supports the treatment effect as well as the safety of the integrin alpha10-selected MSCs as a therapy for ARDS.

FIG. 4: Decreased Damage in Lung Tissue After MSC Treatment

Lung biopsies were taken at upper, middle and lower parts (lobes) of the lungs at the end of the ARDS-study to investigate the degree of damage of the lung tissues and the effect of the MSCs on the lung tissue structure. The results showed less lung tissue damage in MSC-treated animals compared to the control (Non treated) animals demonstrating the therapeutic effect of the MSC in the ARDS model. Representative images in the upper row belong to non-treated group and representative images in the lower row belong to the MSC-treated groups. All images were taken at a magnification of 20X (black scale bars represent 0.1 mm; white scale bar represents 0.2 mm).

FIG. 5: Decreased Neutrophiles in Blood in MSC-Treated Animals.

A) The number of neutrophiles in the blood was analyzed to determine the effect of the integrin α10-selected MSCs on inflammation in the ARDS pigs. The results show decreased numbers of neutrophiles after MSC infusion (Treated) and thus indicate that less neutrophiles are recruited to be part of the inflammation response in the lung tissue in the treated group. This suggests that a decrease in number of neutrophils is one important mechanism of action from the MSCs resulting in lower degree of inflammation and less lung tissue damage. MEAN±S.E.M. p≤0.05.

B) Graph showing number of lymphocytes, million cells per ml, counted in peripheral blood of the animals. In brief, lymphocyte counts in whole blood were elevated at the end of the experiment in the treated group, getting a peak 9 and 10 h after infusion. Lymphocytes are important immune cells involved in response to ARDS and a higher number of lymphocytes could be indicative of a less severe ARDS case.

FIG. 6: Cytokines in Plasma and Bronchoalveolar Lavage Fluid (BALF) Support Immunomodulatory Effect of MSCs

In order to further investigate mechanism of action underlying the immune-modulatory effects of integrin alpha10-selected MSCs in the ARDS model, blood samples and bronchoalveolar lavage fluid (BALF) were taken at different timepoints during the experiment and were analyzed for different relevant inflammatory cytokines. We here demonstrate the effect of the MSCs on Interferon-α (IFN-α) and interleukins IL-12, IL-1β and IL-6 supporting the immunomodulation effect in the MSC-treated animals.

A) The proinflammatory cytokine interferon-α (IFN-α) was detected at higher levels in plasma of the treated animals compared to non-treated animals already 1 h after integrin alpha10-selected MSCs infusion and is sustained for 3 more hours. This higher level can indicate a better prognosis in ARDS as shown by a recent retrospective study showing decrease mortality in patients with ARDS when IFN-α is infused.

B) The proinflammatory cytokine interleukin (IL)-12 was detected at lower levels in the plasma in the integrin alpha10-selected MSCs-treated animals compared to control (Non-treated) animals already 1 h after MSCs infusion, suggesting an immediate effect of the MSCs that is sustained for 8 hours. This further supports the immunomodulatory effect of MSC since this can be indicative of a lower presence of activated antigen presenting cells in blood.

C and D) The pro-inflammatory interleukins IL-1β and IL-6, recognized as key in the development of ARDS, were lower in the BALF in the MSC treated animals compared to the non-treated group after completion of the study (End). Moreover, both IL-1β and IL-6 were significantly increased only in the non-treated animals compared to baseline. This could be indicative of a lower presence of macrophages polarized towards a pro-inflammatory state (M1) and an overall attenuated lung inflammation in treated animals.

### DETAILED DESCRIPTION

### Definitions

“Anti-integrin α10 antibody” or “anti-integrin α10 subunit antibody” is used herein interchangeably to refer to an antibody capable of recognizing and binding to at least the integrin α10 subunit of the heterodimeric protein integrin α10β1. These antibodies may be antibodies that recognize an epitope of the heterodimeric protein integrin α10β1, wherein the epitope comprises amino acid residues of both the integrin α10 and the integrin β1 subunit.

“Integrin α10” or “integrin alpha10” as used herein refers to the α10 subunit of the heterodimeric protein integrin α10β1. This denotation does not exclude the presence of the integrin β1 subunit bound to the integrin α10 subunit thus forming the quaternary structure of integrin α10β1 heterodimer. The human integrin α10 chain sequence is known and publicly available at GenBank™/EBI Data Bank accession number AF074015 and has been described in (Camper 1998). “Alpha” and “a”, as well as “alpha10” and “alpha 10” are equivalent terms.

As used herein, the singular forms “a”, “an” and “the” include plural referents unless the context clearly states otherwise.

The term “some embodiments” can include one, or more than one embodiment.

The use of the word “a” or “an” when used throughout the text or in conjunction with the term “comprising” in the claims and/or the specification may mean “one,” but it is also consistent with the meaning of “one or more,” “at least one,” and “one or more than one.”

The terms “isolating”, “sorting” and “selecting” as used herein refer to the action of identifying a cell as being a certain type of cell and separating it from cells that do not belong to the same cell type or to another differentiation state. Further, these terms may also refer to the action of identifying a cell by the presence of a certain marker. For example, in the present invention in directed to integrin alpha10-selected Mesenchymal Stem Cells (MSCs). Usually, isolation refers to a first step of separation, which may for example be mechanical, whereas “selection” is more specific and for example performed with the help of an antibody. The person skilled in the art will understand that the procedure of “isolating”, “sorting” or “selecting” cells leads to an enrichment of said cells.

The term “integrin alpha10-enriched MSCs” as used herein is synonymous to the terms “integrin alpha 10^{high} MSCs”, “integrin alpha10-selected mesenchymal stem cells” and “an enriched integrin α10^{high} population of mesenchymal stem cells”. As described in Example 1, the MSCs used in the invention are selected using procedures to enrich MSCs expressing integrin alpha10, for example by selecting those MSCs which express integrin alpha10 with the help of an antibody specifically binding to integrin alpha10. The person skilled in the art will understand that cells selected for specific properties, e.g. MSCs expressing integrin alpha10, or integrin alpha 10^{high} MSCs, may form a specific, homogeneous cell population.

“Mesenchymal stem cells” or “MSCs” as used herein refers to multipotent stromal cells as defined by The Mesenchymal and Tissue Stem Cell Committee of the International Society for Cellular Therapy (see Dominici M et al., Cytotherapy. 8(4):315-7 (2006)). MSCs must be plastic-adherent when maintained in standard culture conditions, and must express CD105, CD73 and CD90, and lack expression of CD45, CD34, CD14 or CD11b, CD79alpha or CD19 and HLA-DR surface molecules. MSCs must have the capacity to differentiate to osteoblasts, adipocytes or chondroblasts in vitro.

The terms “disease, disorder or trauma of the respiratory system” as used herein refers to the system that is involved with breathing and refers to any malfunction of one or more parts of the respiratory system. The respiratory system (also referred to as respiratory tract, respiratory apparatus, ventilatory system) includes, for example, the lungs, the bronchi, bronchioles, respiratory bronchioles, alveolar ducts, alveolar sacs and alveoli (sometimes referred to as lower respiratory tract) as well as the trachea, the larynx, the pharynx, the nasal cavity and paranasal sinuses (sometimes referred to as upper respiratory tract.

The term “ARDS (acute respiratory distress syndrome)” as used herein is a life-threatening inflammation, often with oedema in the lungs, which leads to severe respiratory failure. ARDS is a clinical syndrome of lung injury with hypoxic respiratory failure caused by intense, often widespread, pulmonary inflammation that develops after a severe physiologic insult.

The term “sepsis” as used herein refers to a condition defined as “a Systemic Inflammatory Response Syndrome (SIRS) secondary to infection”. Such a condition is characterized by a manifested infection induced by microorganisms, preferably bacteria or fungi, by parasites or by viruses or prions. The term “sepsis” as used herein includes sepsis associated with the final stage of sepsis, and the onset of “severe sepsis”, “septic shock”, and “complications of sepsis” (for example, multiple organ dysfunction syndrome (MODS), disseminated intravascular coagulation (DIC), acute respiratory distress syndrome (ARDS), and acute renal failure (AKI)), and includes all stages of sepsis.

“Preventing” or “Prevention” as used herein, includes delaying or stopping the onset of disease, disorder, or condition.

The terms “disease”, “disorder”, “trauma” and “syndrome” as used herein, and other similar terms, such as “condition”, may be understood as synonyms within this disclosure and refer to a non-functional, pathologic, non-physiologic and/or disturbed state.

### Disease Indications

In one aspect, the present disclosure is directed to a composition comprising integrin alpha10-selected Mesenchymal Stem Cells (MSCs), for use in the treatment of a disease, disorder or trauma of the respiratory system, and/or in connection with transplantation of an organ or tissue of the respiratory tract of a mammal.

In some embodiments of the present disclosure, the disease of the respiratory system is a lower respiratory tract disease.

In some embodiments of the present disclosure, the disease of the respiratory system is a respiratory disease principally affecting the lung interstitium.

In some embodiments of the present disclosure, the disease of the respiratory system is a respiratory disease affecting the airways.

In some embodiments of the present disclosure, the disease of the respiratory system is a disease principally affecting the lung interstitium selected from the group consisting of acute respiratory distress syndrome (ARDS), pulmonary oedema, pulmonary eosinophilia, idiopathic interstitial pneumonitis, primary interstitial lung diseases specific to infancy or childhood, interstitial lung diseases associated with systemic diseases, pulmonary alveolar microlithiasis, lymphangioleiomyomatosis, and lipoid pneumonitis.

As shown in Examples, the herein disclosed integrin alpha10-selected MSCs exhibit anti-inflammatory and immunmodulating properties and can be used to ameliorate, prevent and or treat symptoms related to ARDS. The person skilled in the art will appreciate that diseases or disorders associated with ARDS may also be treated by the herein disclosed integrin alpha10-selected MSCs. For example, diseases or disorders associated with ARDS such as cytokine release syndrome (CRS), cytokine storm syndrome (CSS), and multisystem inflammatory syndrome associated with COVID-19; or complication affecting newborns, for example but not limited to preterm born babies, may affect the respiratory system (e.g. the lungs) and may be treated with the herein disclosed integrin alpha10-selected MSCs. Cytokine storm and cytokine release syndrome are life-threatening systemic inflammatory syndromes involving elevated levels of circulating cytokines and immune-cell hyperactivation that can be triggered, for example, by various therapies, pathogens, cancers, autoimmune conditions, and monogenic disorders.

In some embodiments of the present disclosure, the disease of the respiratory system is acute respiratory distress syndrome (ARDS) and/or associated disorders.

In some embodiments of the present disclosure, the disease of the respiratory system is ARDS.

The disease, disorder or syndrome of the respiratory system that may be treated using the composition disclosed herein may for example be a disorder associated with ARDS.

Thus, in some embodiments of the present disclosure, the disease of the respiratory system is cytokine release syndrome (CRS).

In some embodiments of the present disclosure, the disease of the respiratory system is cytokine storm syndrome (CSS).

In some embodiments of the present disclosure, the ARDS associated disease is multisystem inflammatory syndrome associated with COVID-19.

In some embodiments of the present disclosure, the disease of the respiratory system is cytokine mediated ARDS.

In some embodiments of the present disclosure, the disease of the respiratory system is ARDS/Respiratory distress syndrome of newborn.

In some embodiments of the present disclosure, the disease of the respiratory system is respiratory distress of newborn, such as respiratory distress syndrome of newborn.

The composition for use according to any one of the preceding claims, the disease of the respiratory system is ARDS resulting from trauma.

In some embodiments of the present disclosure, the disease of the respiratory system is ARDS caused by viral or bacterial infection. For example, a viral infection due to severe acute respiratory syndrome coronavirus 2 (SARS-CoV-2) may cause ARDS.

It is appreciated that ARDS can have several underlying causes. However, irrespective of the cause, ARDS is a recogniced clinical syndrome defined by clinical parameters, for example severe hypoxemia despite administration of supplemental oxygen, bilateral pulmonary infiltrates and reduced lung compliance. Several measurable factors, for example cytokines, are suspected or known to be involved in the pathology of ARDS. The person skilled in the art will understand that integrin alpha10-selected Mesenchymal Stem Cells (MSCs) may be used in the treatment of ARDS triggered by different kind of underlying causes.The herein included examples illustrate a recognized animal model for ARDS, and the person skilled in the art will understand that other models known in the art can be used to evaluate the efficacy of the herein disclosed MSCs.

A frequent cause of ARDS is sepsis, and ARDS is in many cases the condition which ultimately leads to the death of the patient suffering from sepsis. Consequenly, since the herein disclosed integrin alpha10-selected Mesenchymal Stem Cells (MSCs) show efficacy in treating ARDS, said cells may be used to treat the underlying cause of ARDS, for example sepsis.

Another cause of ARDS is evere acute respiratory syndrome coronavirus 2 (SARS-CoV-2)/COVID-19, and ARDS may be the condition which ultimately leads to the death of the patient suffering from COVID-19. Consequenly, since the herein disclosed integrin alpha10-selected Mesenchymal Stem Cells (MSCs) show efficacy in treating ARDS, said cells may be used to treat the underlying cause of ARDS, for example COVID-19.

Thus, in some embodiments of the present disclosure, the disease of the respiratory system is ARDS resulting from severe acute respiratory syndrome coronavirus 2 (SARS-CoV-2)/COVID-19.

In some embodiments of the present disclosure, the disease of the respiratory system is ARDS resulting from any other cause.

Disease mechanisms involved in ARDS, such as the increase of proinflammatory cytokines and cellular changes, especially relating to immune cells, may also be involved in mechanisms involved in rejection of a transplanted organ or tissue, for example in the setting of lung transplantion. Thus, the herein disclosed composition comprising integrin alpha10-selected Mesenchymal Stem Cells (MSCs) may be used to prevent or treat disorders in connection with transplantation of an organ, for example lung transplantation.

Thus, some of the embodiments of the present disclosure relate to the use of the composition comprising integrin alpha10-selected Mesenchymal Stem Cells (MSCs) in the treatment of a disease, disorder or trauma in connection with transplantation of an organ or tissue of the respiratory tract of a mammal, as for example lung transplantation.

### MSC Characteristics and Manufacture

In some embodiments of the present disclosure, at least 60% of the MSCs express integrin α10 subunit.

Example 1 descibes a way of manufacturing the integrin alpha10 selected MSCs presented in the present disclosure. The key advantage of the integrin alpha10 selected MSCs is that they have been selected using the criteria of integrin alpha10 protein expression and are thus an homogeneous culture and/or population of MSCs. These cells have been shown to exibit robust expression of stem cell markers, see for example WO 2018/138322. The skilled person in the art will know that several methods for selecting, and thereby enriching cells, can be used. In the present invention, integrin alpha10 high MSCs are enriched during the isolation/selection procedure. For this, an anti-integrin alpha10 antibody may be used. MSC isolation and selection may be perfomed as described in WO 2018/138322.

As disclosed in Example 1, at the selection stage the selected MSCs, which are selected by their expression of integrin alpha10 using an anti-integrin alpha 10 antibody, express integrin alpha10 (Integrin alpha10 expressing MSCs may be referred to as Integrin alpha10^{high} MSCs). More specifically, the selected cells are MSCs which express the heterodimer integrin alpha10 beta1(α10β1), since the integrin alpha10 subunit is expressed together with the integrin beta1 subunit. The selection stage is followed by an expansion stage where integrin alpha10 expression of each of the selected MSCs may vary, i.e. not all MSCs may express integrin alpha10 at all time during expansion and thus at the time of administration. However, at the time of administering the MSCs to a patient, at least 50% of the administered cells express integrin alpha10 subunit.

In some embodiments of the present disclosure, at least 50%, such as at least 55%, such as at least 60%, such as at least 65%, such as at least 70%, such as at least 75%, such as at least 80%, such as at least 85%, such as at least 90%, such as at least 95%, such as at least 96%, such as at least 97%, such as at least 98%, such as at least 99%, such as at least 100% of the MSCs express integrin alpha10 subunit.

In some embodiments of the present disclosure, the MSCs are MHC class II, CD45, CD34, CD11b and/or CD19 negative.

In some embodiments of the present disclosure, the MSCs express CD73, CD90 and/or CD105.

In some embodiments of the present disclosure, the MSCs are selected from the group consisting of a mesenchymal stem cells, mesenchymal progenitor cells, and mesenchymal stromal cells; or a mixture thereof.

In some embodiments of the present disclosure, the MSCs are induced to express integrin α10 subunit.

In some embodiments of the present disclosure, the MSCs are cultured in a culture media comprising mammalian serum and FGF-2.

In some embodiments of the present disclosure, the MSCs are cultured in a culture media comprising platelet lysate and/or platelet lysate components.

In some embodiments of the present disclosure, the MSCs are cultured in a culture media comprising FGF-2 and platelet lysate and/or platelet lysate components.

In some embodiments of the present disclosure, the MSCs are cultured in a culture media comprising mammalian serum and platelet lysate and/or platelet lysate components.

In some embodiments of the present disclosure, the MSCs are cultured in a culture media comprising TGFβ.

In some embodiments of the present disclosure, the MSCs are cultured in a culture media comprising FGF2.

In some embodiments of the present disclosure, the MSCs are cultured in a serum-free culture media comprising platelet lysate and/or platelet lysate components.

In some embodiments of the present disclosure, the MSCs are cultured in a serum-free culture media comprising growth factors.

In some embodiments of the present disclosure, the MSCs are cultured in a serum-free culture media comprising the growth factors FGF2 and/or TGFβ.

In some embodiments of the present disclosure, the MSCs are allogeneic or autologous.

In some embodiments of the present disclosure, the MSCs and mammal are from the same species.

In some embodiments of the present disclosure, the MSCs and mammal are from different species.

In some embodiments of the present disclosure, the MSCs are derived from adipose tissue, bone marrow, synovial membrane, peripheral blood, cord blood, umbilical cord blood, Wharton's jelly, and/or amniotic fluid.

The person skilled in the art with appreciate that the herein disclosed procedure for selecting integrin alpha10-selected MSCs would also be applicable to select MSCs from other sources known in the art.

In some embodiments of the present disclosure, the MSCs are derived from adipose tissue.

In some embodiments of the present disclosure, the MSCs are derived from bone marrow.

In some embodiments of the present disclosure, the MSCs are derived from fetal, neonatal, juvenile or adult MSCs and/or progenitor cells.

In some embodiments of the present disclosure, the MSCs are not derived from embryonic cells or from an embryo.

In some embodiments of the present disclosure, the MSCs are an in vitro cell culture.

In some embodiments of the present disclosure, the selection of MSCs has been conducted with an anti-integrin α10 antibody.

### Administration

In some embodiments of the present disclosure, the composition comprising the integrin alpha10-selected MSCs is administered into the lung or airways.

In some embodiments of the present disclosure, wherein the composition comprising the integrin alpha10-selected MSCs is administered via injection.

The person skilled in the art will know of other ways known in the art for administration of integrin alpha10-selected MSCs.

In some embodiments of the present disclosure, the composition comprising the integrin alpha10-selected MSCs is administered parenterally.

Accordingly, the composition comprising integrin alpha10-selected MSCs for use according to the present disclosure may be administered topically to cross any mucosal membrane of an animal to which the integrin alpha10-selected MSCs is to be given.

In some embodiments of the present disclosure, the composition comprising the integrin alpha10-selected MSCs is administered via intravenous injection, intramuscular injection and/or intratracheal injection, or any combination thereof.

In some embodiments of the present disclosure, the integrin alpha10-selected MSCs are formulated into a cell aggregate prior to administration.

In some embodiments of the present disclosure, the composition comprising the integrin alpha10-selected MSCs is administered in a cell suspension with a pharmaceutically acceptable excipient.

In some embodiments of the present disclosure, the composition comprising the integrin alpha10-selected MSCs is administered during surgery to repair a damaged lung.

In some embodiments of the present disclosure, the composition comprising the integrin alpha10-selected MSCs is administered in connection with lung transplantation.

In some embodiments of the present disclosure, the mammal is a human.

In some embodiments of the present disclosure, the mammal is a human, horse, pony, ox, donkey, mule, camelid, cat, dog, pig, or cow.

In some embodiments of the present disclosure, the integrin alpha10-selected MSCs and mammal are from the same species.

In some embodiments of the present disclosure, the integrin alpha10-selected MSCs and mammal are from different species.

In some embodiments of the present disclosure, the integrin alpha10-selected MSCs are derived from adipose tissue, bone marrow, synovial membrane, peripheral blood, cord blood, umbilical cord blood, Wharton's jelly, and/or amniotic fluid.

In some embodiments of the present disclosure, the integrin alpha10-selected MSCs are derived from adipose tissue.

In some embodiments of the present disclosure, the integrin alpha10-selected MSCs are derived from bone marrow.

In some embodiments of the present disclosure, the integrin alpha10-selected MSCs are derived from fetal, neonatal, juvenile or adult MSCs and/or progenitor cells.

In some embodiments of the present disclosure, the integrin alpha10-selected MSCs are not derived from embryonic cells or from an embryo.

In some embodiments of the present disclosure, the integrin alpha10-selected MSCs are an in vitro cell culture.

In some embodiments of the present disclosure, the selection of integrin alpha10-selected MSCs has been conducted with an anti-integrin α10 antibody.

In some embodiments of the present disclosure, the composition comprising integrin alpha10-selected Mesenchymal Stem Cells (MSCs), further comprises an anti-inflammatory and/or immunomodulatory agent.

In another aspect, the present disclosure is directed to the use of a composition comprising integrin α10-selected Mesenchymal Stem Cells (MSCs), for the manufacture of a medicament for the treatment of a disease, disorder or trauma of the respiratory system, and/or in connection with transplantation of an organ or tissue of the respiratory tract of a mammal.

### Methods

In a further aspect, the present disclosure is directed to a method of treatment of a disease, disorder or trauma of the respiratory system of a mammal, the method comprising administering a therapeutically effective amount of a composition comprising integrin α10-selected Mesenchymal Stem Cells (MSCs).

In a further aspect, the present disclosure is directed to a method of treatment or promotion of a transplantation of an organ or tissue of the respiratory tract of a mammal, the method comprising administering a therapeutically effective amount of a composition comprising integrin α10-selected Mesenchymal Stem Cells (MSCs).

In a further aspect, the present disclosure is directed to a method of preventing blood clotting in a mammal in connection with a disease, disorder, or trauma of the respiratory system, and/or in connection with transplantation of an organ or tissue of the respiratory tract of a mammal, the method comprising administering a therapeutically effective amount of a composition comprising integrin α10-selected Mesenchymal Stem Cells (MSCs).

In a further aspect, the present disclosure is directed to a method of promoting hemodynamic stability in connection with a disease, disorder, or trauma of the respiratory system, and/or in connection with transplantation of an organ or tissue of the respiratory tract of a mammal, the method comprising administering a therapeutically effective amount of a composition comprising integrin α10-selected Mesenchymal Stem Cells (MSCs).

In a further aspect, the present disclosure is directed to a method of reducing the need for inotropic support in connection with a disease, disorder, or trauma of the respiratory system, and/or in connection with transplantation of an organ or tissue of the respiratory tract of a mammal, the method comprising administering a therapeutically effective amount of a composition comprising integrin α10-selected Mesenchymal Stem Cells (MSCs).

In a further aspect, the present disclosure is directed to a method of improving oxygenation capacity in connection with a disease, disorder, or trauma of the respiratory system, and/or in connection with transplantation of an organ or tissue of the respiratory tract of a mammal, the method comprising administering a therapeutically effective amount of a composition comprising integrin α10-selected Mesenchymal Stem Cells (MSCs).

In a further aspect, the present disclosure is directed to a method of preventing tissue damage, for example structural tissue damage, in connection with a disease, disorder, or trauma of the respiratory system, and/or in connection with transplantation of an organ or tissue of the respiratory tract of a mammal, the method comprising administering a therapeutically effective amount of a composition comprising integrin α10-selected Mesenchymal Stem Cells (MSCs).

In a further aspect, the present disclosure is directed to a method of reverting tissue damage, for example structural tissue damage, in connection with a disease, disorder, or trauma of the respiratory system, and/or in connection with transplantation of an organ or tissue of the respiratory tract of a mammal, the method comprising administering a therapeutically effective amount of a composition comprising integrin α10-selected Mesenchymal Stem Cells (MSCs).

In some embodiments of the present disclosure, the tissue damage to be prevented or reverted by the disclosed method is lung tissue damage.

In some embodiments of the present disclosure, the tissue damage to be prevented or reverted by the disclosed method is damage of the interstitial tissue, damage of the alveolar septa, damage of the airways, damage of the vasculature and/or damage of the nervous system.

In a further aspect, the present disclosure is directed to a method of reducing neutrophil counts in connection with a disease, disorder, or trauma in connection with a disease, disorder, or trauma of the respiratory system, and/or in connection with transplantation of an organ or tissue of the respiratory tract of a mammal, the method comprising administering a therapeutically effective amount of a composition comprising integrin α10-selected Mesenchymal Stem Cells (MSCs).

In a further aspect, the present disclosure is directed to a method of increasing lymphocyte counts in connection with a disease, disorder, or trauma in connection with a disease, disorder, or trauma of the respiratory system, and/or in connection with transplantation of an organ or tissue of the respiratory tract of a mammal, the method comprising administering a therapeutically effective amount of a composition comprising integrin α10-selected Mesenchymal Stem Cells (MSCs).

In a further aspect, the present disclosure is directed to a method of decreasing proinflammatory cytokines in connection with a disease, disorder, or trauma in connection with a disease, disorder, or trauma of the respiratory system, and/or in connection with transplantation of an organ or tissue of the respiratory tract of a mammal, the method comprising administering a therapeutically effective amount of a composition comprising integrin α10-selected Mesenchymal Stem Cells (MSCs). In some embodiments of the present disclosure, the proinflammatory cytokines to be decreased by the disclosed method are selected from the group consisting of interleukin 12 (IL-12), IL-1β, IL-6 and IL-4, or any combination thereof.

In some embodiments of the present disclosure, the proinflammatory cytokines to be decreased by the disclosed method are decreased in blood and/or bronchoalveolar lavage fluid.

In a further aspect, the present disclosure is directed to a method of increasing interferon-α in connection with a disease, disorder, or trauma in connection with a disease, disorder, or trauma of the respiratory system, and/or in connection with transplantation of an organ or tissue of the respiratory tract of a mammal, the method comprising administering a therapeutically effective amount of a composition comprising integrin α10-selected Mesenchymal Stem Cells (MSCs).

In a further aspect, the present disclosure is directed to a composition comprising integrin alpha10-selected Mesenchymal Stem Cells (MSCs), for use in a method of
preventing blood clotting,
promoting hemodynamic stability,
reducing the need for inotropic support,
improving oxygenation capacity,
preventing tissue damage, for example structural tissue damage,
reverting tissue damage, for example structural tissue damage,
reducing neutrophil counts,
increasing lymphocyte counts,
decreasing proinflammatory cytokines, and/or
increasing interferon-α,
in a mammal in connection with a disease, disorder, or trauma of the respiratory system, and/or in connection with transplantation of an organ or tissue of the respiratory tract of a mammal.

Some embodiments of the present disclosure are directed to a composition comprising integrin alpha10-selected Mesenchymal Stem Cells (MSCs), for use in a method of preventing blood clotting in a mammal in connection with a disease, disorder, or trauma of the respiratory system, and/or in connection with transplantation of an organ or tissue of the respiratory tract of a mammal.

Some embodiments of the present disclosure are directed to a composition comprising integrin alpha10-selected Mesenchymal Stem Cells (MSCs), for use in a method of promoting hemodynamic stability in a mammal in connection with a disease, disorder, or trauma of the respiratory system, and/or in connection with transplantation of an organ or tissue of the respiratory tract of a mammal.

Some embodiments of the present disclosure are directed to a composition comprising integrin alpha10-selected Mesenchymal Stem Cells (MSCs), for use in a method of reducing the need for inotropic support in a mammal in connection with a disease, disorder, or trauma of the respiratory system, and/or in connection with transplantation of an organ or tissue of the respiratory tract of a mammal.

Some embodiments of the present disclosure are directed to a composition comprising integrin alpha10-selected Mesenchymal Stem Cells (MSCs), for use in a method of improving oxygenation capacity in a mammal in connection with a disease, disorder, or trauma of the respiratory system, and/or in connection with transplantation of an organ or tissue of the respiratory tract of a mammal.

Some embodiments of the present disclosure are directed to a composition comprising integrin alpha10-selected Mesenchymal Stem Cells (MSCs), for use in a method of preventing tissue damage, for example structural tissue damage in a mammal in connection with a disease, disorder, or trauma of the respiratory system, and/or in connection with transplantation of an organ or tissue of the respiratory tract of a mammal.

Some embodiments of the present disclosure are directed to a composition comprising integrin alpha10-selected Mesenchymal Stem Cells (MSCs), for use in a method of reverting tissue damage, for example structural tissue damage, in a mammal in connection with a disease, disorder, or trauma of the respiratory system, and/or in connection with transplantation of an organ or tissue of the respiratory tract of a mammal.

Some embodiments of the present disclosure are directed to a composition comprising integrin alpha10-selected Mesenchymal Stem Cells (MSCs), for use in a method of reducing neutrophil counts in a mammal in connection with a disease, disorder, or trauma of the respiratory system, and/or in connection with transplantation of an organ or tissue of the respiratory tract of a mammal.

Some embodiments of the present disclosure are directed to a composition comprising integrin alpha10-selected Mesenchymal Stem Cells (MSCs), for use in a method of increasing lymphocyte counts in a mammal in connection with a disease, disorder, or trauma of the respiratory system, and/or in connection with transplantation of an organ or tissue of the respiratory tract of a mammal.

Some embodiments of the present disclosure are directed to a composition comprising integrin alpha10-selected Mesenchymal Stem Cells (MSCs), for use in a method of decreasing proinflammatory cytokines in a mammal in connection with a disease, disorder, or trauma of the respiratory system, and/or in connection with transplantation of an organ or tissue of the respiratory tract of a mammal.

Some embodiments of the present disclosure are directed to a composition comprising integrin alpha10-selected Mesenchymal Stem Cells (MSCs), for use in a method of increasing interferon-α in a mammal in connection with a disease, disorder, or trauma of the respiratory system, and/or in connection with transplantation of an organ or tissue of the respiratory tract of a mammal.

In some embodiments of the present disclosure, the tissue damage is lung tissue damage.

In some embodiments of the present disclosure, the tissue damage is damage of the interstitial tissue, damage of the alveolar septa, damage of the airways, damage of the vasculature, damage of muscle and/or damage of the nervous system. The method according to claim 69, wherein the proinflammatory cytokines are selected from the group consisting of interleukin 12 (IL-12), IL-1β, IL-6 and IL-4, or any combination thereof.

In some embodiments of the present disclosure, the proinflammatory cytokines are decreased in blood and/or bronchoalveolar lavage fluid.

### EXAMPLES

### Example 1: Production of Integrin Alpha10-Enriched MSCs

### Aim

This example illustrates how integrin alpha10-selected MSCs are isolated, selected, expanded and stored until usage in the treatment model.

### Material and Methods:

Integrin alpha10-selected mesenchymal stem cells (MSCs) were isolated from human or animal adipose donor tissue or from other MSC-containing sources. The adipose tissue was dissociated/digested and the adipose-derived stromal vascular fraction (SVF) was re-suspended in MSC expansion medium and seeded into cell culture flasks to allow the MSCs to adhere to the plastic and proliferate.

The plastic-adherent cells were analyzed for positive expression (≥95%) of the cell surface markers CD73, CD90 and CD105, and negative expression (≤2%) of CD45, CD34, CD11b, CD19 and HLA-DR, as measured by flow cytometry. This specific antigen expression criteria is also a part of the MSC definition set by the International Society for Cellular Therapy (Dominici 2006). The MSC-preparation was expanded in monolayer cultures in MSC expansion medium and Integrin alpha10-expressing MSCs were selected using antibodies specifically binding to integrin alpha10 (thereby recognizing the full receptor integrin alpa10 beta1, i.e. integrin α10β1) and magnetic bead separation or selected by FACS cell sorting. The integrin alpha10-selected MSC were further expanded, checked for cell surface expression of the defined MSC antigens and in addition, trilineage differentiation capability was demonstrated. The alpha10-selected MSCs were frozen live in cryopreservation medium and kept frozen until use.

### Results:

The procedure resulted in integrin alpha10-selected MSCs, expanded and frozen in vials which can be used for administration, for example i. v. administration.

### Conclusions

The manufacturing process generates alpha10-selected MSCs fulfilling the minimal criteria defining human MSCs and could be applied in cell therapy.

### Example 2: Demonstrating Efficacy and Safety of Integrin Alpha10-Selected MSC Treatment in a Porcine ARDS Model

### Aim

The aim of these experiments was to demonstrate and evaluate the therapeutic effect of the integrin alpha10-selected MSCs on ARDS in a validated porcine model as well as the safety of the intraarterial infusion of integrin alpha10-selected MSCs.

### Material and Methods

Twelve pigs with a mean weight of 35.83±4.79 kg were used. General anaesthesia was administered, and a peripheral intravenous catheter was placed in the earlobe, together with an endotracheal intubation and mechanical ventilaton with nonhumidified air. Ventilation was adjusted to maintain carbon dioxide levels (PaCO₂) between 33 and 41 mmHg.

To induce ARDS, lipopolysaccharide (LPS) from gram-negative bacteria Escherichia coli was diluted and administered via endotracheal (ET) installation and via pulmonary artery.

Hemodynamics, gas exchange, need of inotropic support, need of fluid replacement, urine output, cytokine response and coagulation cascade response in plasma were monitored continuously throughout the study period. Lung tissue for RNA sequencing and immunohistochemical analysis was collected.

Hemodynamic parameters, blood gases and blood clotting time (using a ROTEM instrument) were measured to confirm and monitor the porcine ARDS model and assess the clinical effect of the infused integrin alpha10-selected MSCs. The different

ARDS stages were defined according to the Berlin definition using the partial pressure of oxygen (PaO₂)-to-fraction of inspired oxygen FIO₂ ratio. After establishment of ARDS the pigs were randomized to either integrin alpha10-selected MSC treatment (5 million MSC/kg, given intravenously) or sham treatment cryomedium containing 5-10% DMSO. Lung biopsies were taken from the right lobe at the termination of the experiment through sternotomy from all lobes. Hematoxylin and eosin (H&E)-stained lung biopsies were used to confirm the onset of severe lung damage and the effect of the infused MSCs on preserving lung integrity in our model.

### Results

### Analysis of Hemodynamic Stability/Need of Inotropic Support

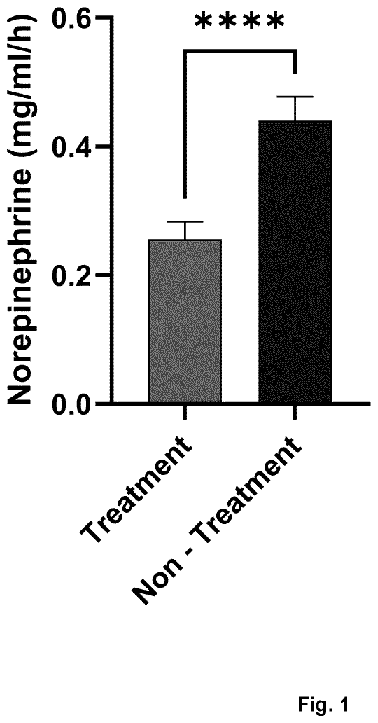

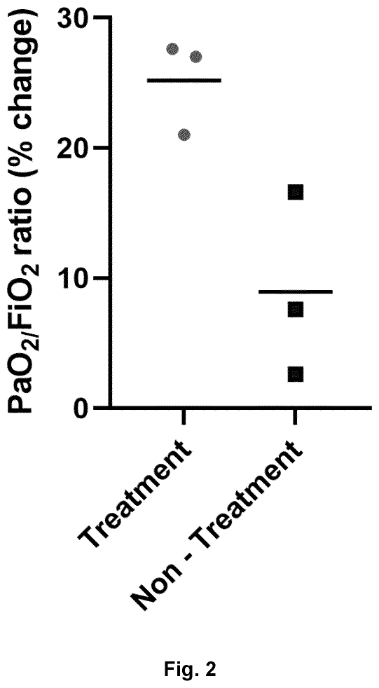

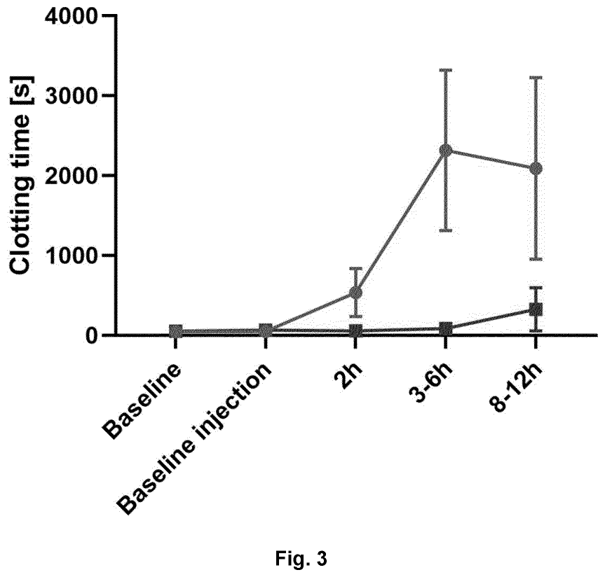

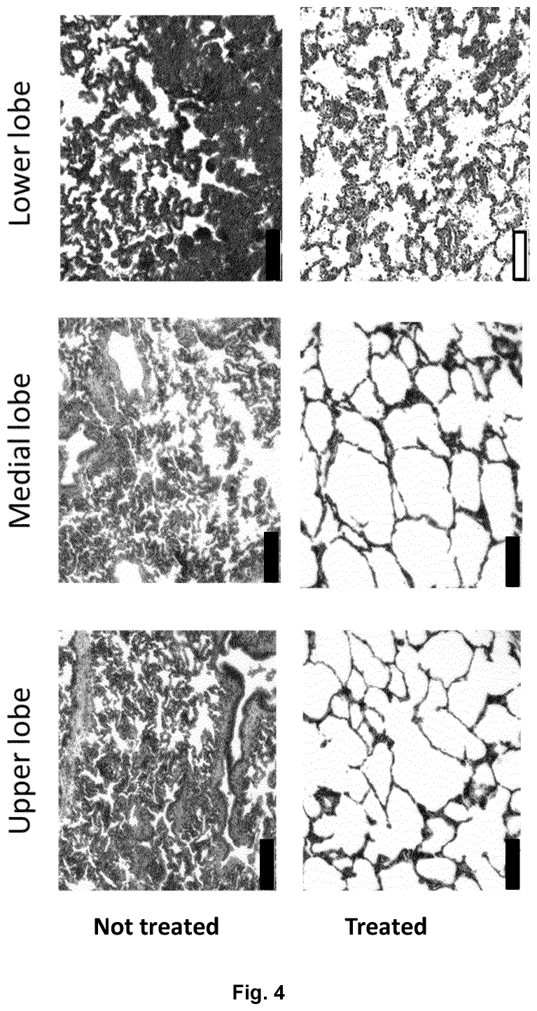

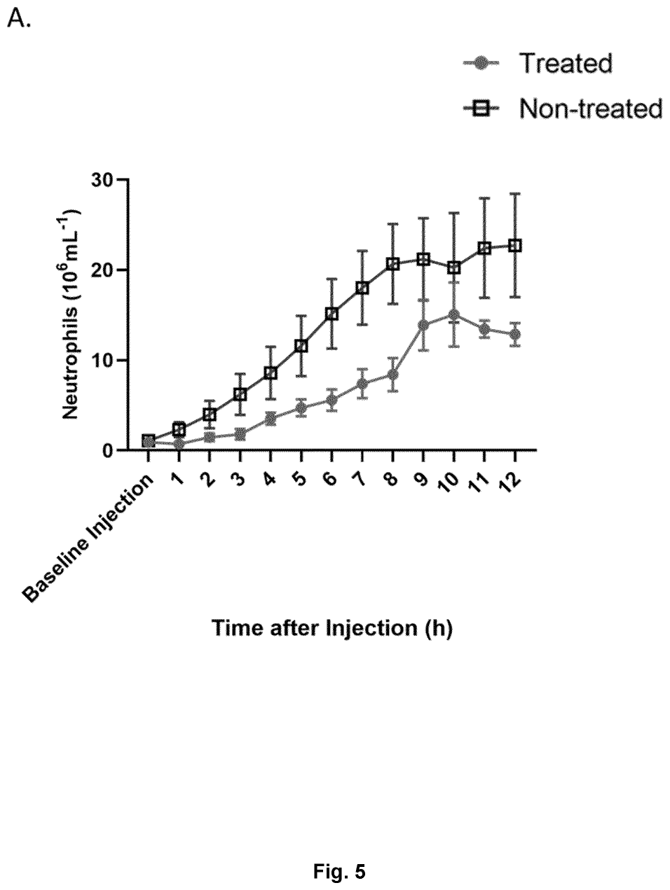

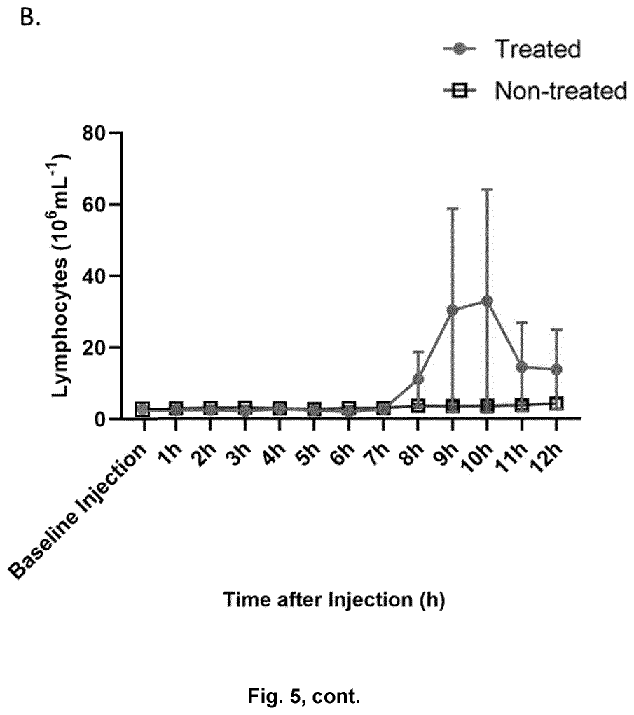

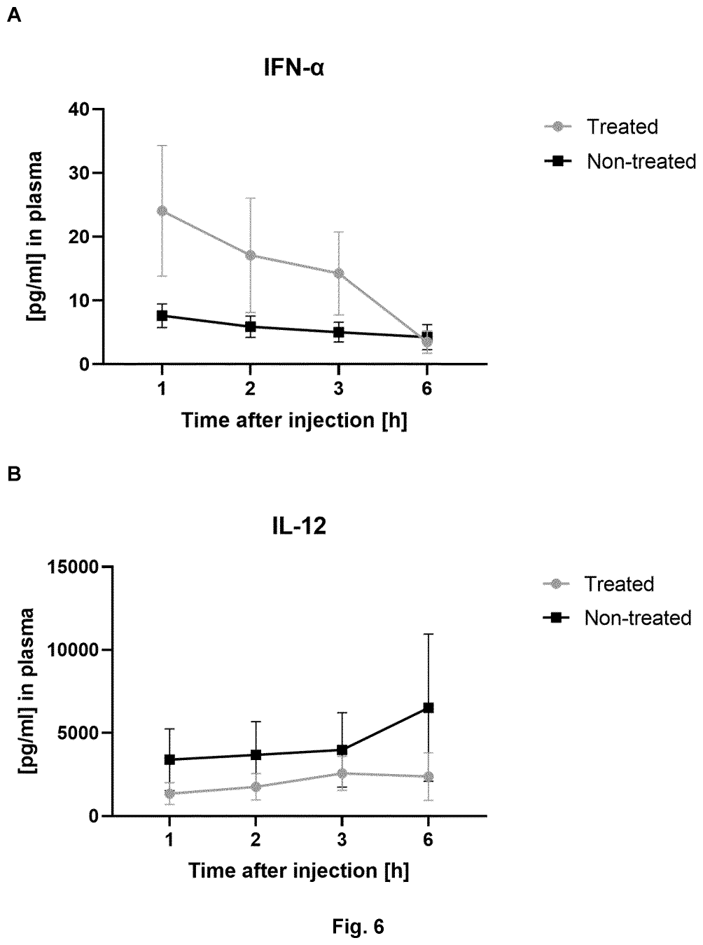

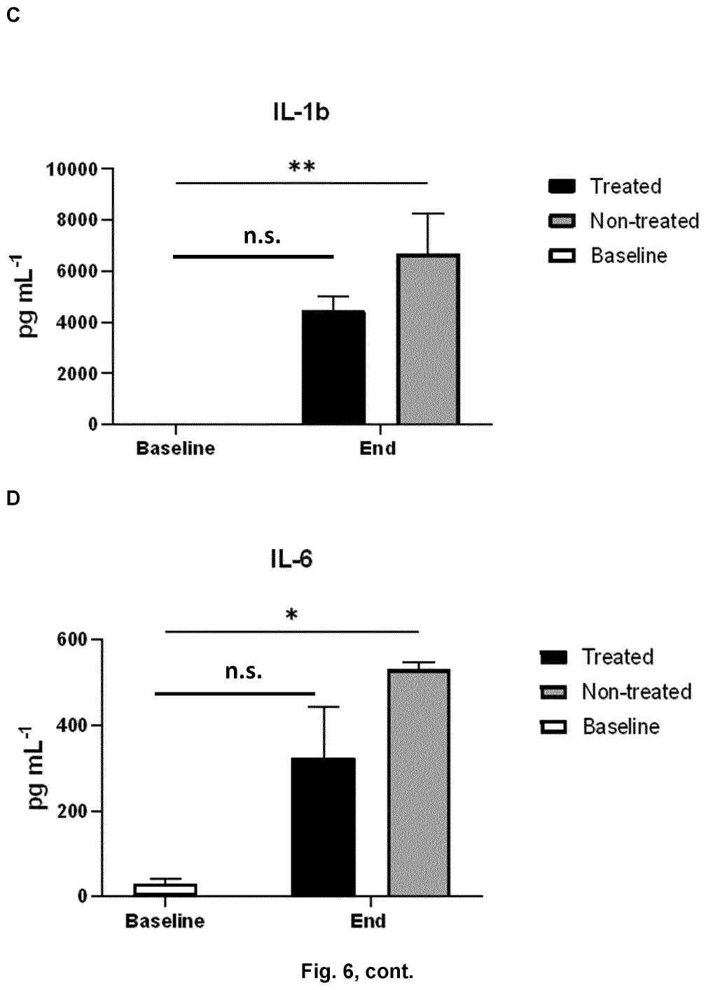

Inotropic support such as administration of norepinephrine is given for ensuring hemodynamic stability and oxygenation levels in the ARDS model during the course of experiment. We found that the total amount of administrated norepinephrine was significantly less in the integrin alpha10-selected MSC-treated animals compared to the control group indicating that animals treated with the integrin alpha10-selected MSCs have more stable hemodynamic parameters and can ensure a better oxygen distribution (FIG. 1).

### Analysis of Oxygenation Capacity

In line with less administration of norepinephrine, blood gas values also confirmed that the oxygenation capacity was elevated in the integrin alpha10-selected MSC-treated ARDS animals. We were able to analyse oxygenation in three integrin alpha10-selected MSC-treated and three control animals after 12 hours. We found that the integrin alpha10-selected MSC-treated animals had improved oxygenation capacity compared to the control animals supporting the treatment effect of the integrin alpha10-selected MSCs (FIG. 2).

### Analysis of Coagulation Time

Coagulation of the blood and formation of blood clots is a common and huge medical problem in ARDS. We thus investigated the effect of the infused integrin alpha10-selected MSCs on the coagulation (blood clotting) time during the course of the ARDS study. We found a significant decrease in clot time in the integrin alpha10-selected MSC-treated animals compared to the control animals. The effect was seen already after 2 hours and was pronounced from 3 hrs and forward (FIG. 3). The dramatic reduction of blood clot time formation is an important efficacy parameter and also demonstrated the safety of administrating the integrin alpha10-selected MSCs intravenously. These results support the infusion of integrin alpha10-selected MSCs as an effective treatment for prevention of clot formation in ARDS, one of the main hallmarks of this pathology (Frantzeskaki 2017).

### Histology

Hematoxylin and eosin (H&E)-staining of lung tissue sections of the lungs from the upper, medial and lower lobes were analysed to compare the lung tissue structure between integrin alpha10-selected MSC-treated and non-treated pigs. We found severe destruction of the lung tissue in the control animals and significant less damage to the lung tissue and much better-preserved lung structure in the integrin alpha10-selected MSC-treated group (FIG. 4). The results are in line with the above-mentioned clinical results for the hemodynamics and the oxygenation capacity and clearly demonstrate the therapeutic effect of the integrin alpha10-selected MSCs in the ARDS model.

### Conclusions

By comparing integrin alpha10-selected CS-treated pigs with placebo-treated pigs the safety and efficacy of integrin alpha10-selected MSCs in an animal model was established. We have found that integrin alpha10-selected MSCs have a treatment effect on ARDS in a clinically relevant porcine ARDS model. The intravenously administrated integrin alpha10-selected MSCs were able to impove hemodynamics, lung oxygenation, decrease blood clot formation and preserve the integrity of the lung tissue structure.

### Example 3: Demonstrating Anti-Inflammatory and Immune-Modulatory Effects of Integrin Alpha10-Selected MSCs in the Porcine ARDS Model

### Aim

To investigate the mechanism of action underlying the treatment effects of integrin alpha10-selected MSCs in the porcine ARDS model. For example, anti-inflammatory and/or immune-modulatory effects were investigated.

### Material and Methods

Blood samples were collected from the integrin alpha10-selected MSC-treated pigs and control pigs at different time points during the ARDS study to analyse number of neutrophils and concentration of different pro-inflammatory and anti-inflammatory cytokines in the blood plasma. Analysis of cytokine levels in plasma and broncho-alveolar lavage (BAL) at different time points was conducted using a multiplex immunoassay kit that measures 9 cytokines. Analyzing the immunophenotype seen in Peripheral Blood Mononuclear Cells (PBMCs) at different time points may mirror the cytokine profile and might hence correlate with the “clinical” outcome during the study. Biopsies from the lung could be used to generate insights of the pathophysiology and its compartment infiltrating cells. Bronchoalveolar lavage fluid (BALF) samples were also collected at the start and at the termination of the study. Number of neutrophils and lymphocytes were analysed by Sysmex and concentration of different cytokines were measured by multiplex assays using cytokine-specific antibodies, Luminex.

### Results

### Analysis of Neutrophil and Lymphocyte Cell Counts

The number of neutrophiles and lymphocytes were analysed in blood samples at different time points during the ARDS study. We found that neutrophil counts were lower in the MSC-treated animals compared to control animals suggesting an immunomodulatory and anti-inflammatory effect of the integrin alpha10-selected MSCs. A difference between integrin alpha10-selected MSC-treated and non-treated animals was seen already after 1 hour and then this difference increased with time (FIG. 5A). Analysis of lymphocyte counts showed an increase of lymphocytes in the integrin alpha10-selected MSC-treated animals after 8 hours of treatment but not in the non-treated animals (FIG. 5B). This finding is supported by a retrospective analysis of ARDS patients, where higher lymphocytes count correlated with higher survival rates (Song 2020).

Analysis of Proinflammatory Cytokines from Blood and from BALF

Several pro-inflammatory cytokines including interleukin 12 (IL-12), IL-1β and IL-6 were detected at lower concentration the blood plasma in the integrin alpha10-selected MSC treated animals compared to the control animals. The difference was seen already 1 h after the infusion of integrin alpha10-selected MSCs, suggesting an immediate immunomodulatory effect of the integrin alpha10-selected MSCs. The proinflammatory cytokine interleukin (IL)-12 was detected at lower levels in the plasma in the integrin alpha10-selected MSCs-treated animals compared to control animals already 1 h after MSCs infusion, suggesting an immediate effect of the MSCs that is sustained for at least 6 hours (FIG. 6B). This further supports the immunomodulatory effect of MSCs since this can be indicative of a lower presence of activated antigen presenting cells in blood (Dorman 2000). Furthermore, the levels of interferon-α (IFN-α) were elevated (FIG. 6A) in the integrin alpha10-selected MSC treated animals compared to the non-treated animals after 1 h and sustained for several hours after integrin alpha10-selected MSC infusion.

Interestingly, elevated levels of IFN-α have been shown to be associated with better prognosis of the disease in ARDS patients (Wang 2020).

We also analysed levels of cytokines in BALF at the end of the study. The results showed that BALF pro-inflammatory cytokines IL-1β (IL-1b) and IL-6 were significantly increased in the non-treated animals a opposed to the treated animals (FIG. 6 C, D). This could be indicative of a lower presence of macrophages polarized towards a pro-inflammatory state and an overall attenuated lung inflammation (McGonagle 2020).

By studying inflammatory markers and the profile of the immune response in treated vs. non-treated animals, important insight in the mechanisms underlying anti-inflammatory or immune-modulatory effects of integrin alpha10-selected MSCs were gained. It will be understood that the results of this study as presented in Examples 2 and 3 can be confirmed in larger pigs as well, e.g. pigs of 60-70 kg, and in other established models of ARDS and associated disorders.

### Conclusions:

We found that infusion of integrin alpha10-selected MSCs in a porcine model for severe ARDS resulted in decreased number of neutrophils in the blood and decreased levels of proinflammatory cytokines in blood plasma and BALF. This suggesst that integrin alpha10-selected MSCs have anti-inflammatory and immunomodulatory effects in the ARDS model which likely represents mechanmism of actions involved in improved hemodynamics and preserved lung integrity and function seen in the integrin alpha10-selected MSC-treated animals. A lower level of circulating cytokines in plasma indicates a lower risk for developing a cytokine storm, which is a feature of ARDS (Hojyo 2000) and thus a less severe ARDS progression in the integrin alpha10-selected MSC-treated animals.

### References

Camper, Hellman, Lundgren-Akerlund; J Biol Chem. 1998 Aug. 7;273(32):20383-9; Isolation, cloning, and sequence analysis of the integrin subunit alpha10, a beta1-associated collagen binding integrin expressed on chondrocytes.
Casey, Semler, Rice; Semin Respir Crit Care Med. 2019 February;40(1):57-65; Fluid Management in Acute Respiratory Distress Syndrome.
Dominici, M., Le Blanc, K., Mueller, I., Slaper-Cortenbach, I., Marini, F. C., and Krause, D. S. (2006). Minimal criteria for defining multipotent mesenchymal stromal cells. The international society for cellular therapy position statement. Cytotherapy 8, 315-317.
Dorman, Holland; Cytokine Growth Factor Rev. 2000 December;11(4):321-33; Interferon-gamma and interleukin-12 pathway defects and human disease.
Frantzeskaki, Armaganidis, Orfanos; Respiration. 2017;93(3):212-225. doi: 10.1159/000453002. Epub 2016 Dec. 21, lmmunothrombosis in Acute Respiratory Distress Syndrome: Cross Talks between Inflammation and Coagulation.
Gonzales, Lucas, Verin; Austin J Vasc Med. 2015 Jun. 4;2(1):1009; The Acute Respiratory Distress Syndrome: Mechanisms and Perspective Therapeutic Approaches.
Hojyo, Uchida, Tanaka, Hasebe, Tanaka, Murakami and Hirano; Cytokine Growth Factor Rev. 2000 December;11(4):321-33; Interferon-gamma and interleukin-12 pathway defects and human disease.
Matthay; Nat Rev Dis Primers. 2019 Mar. 14;5(1):18; Acute respiratory distress syndrome.
McGonagle, Sharif, O'Regan, Bridgewood; Autoimmun Rev; 2020 June;19(6):102537. doi: 10.1016/j.autrev.2020.102537. The Role of Cytokines including Interleukin-6 in COVID-19 induced Pneumonia and Macrophage Activation Syndrome-Like Disease.
Prescott; Am J Respir Crit Care Med. 2016 Jul. 15;194(2):147-55; Toward Smarter Lumping and Smarter Splitting: Rethinking Strategies for Sepsis and Acute Respiratory Distress Syndrome Clinical Trial Design.
Song, Liu, Lu, Luo, Peng, Chen; BMC Pulm Med. 2020 Apr. 23;20(1):102; Prognostic factors for ARDS: clinical, physiological and atypical immunodeficiency.
Wang, N. et al. Retrospective multicenter cohort study shows early interferon therapy is associated with favorable clinical responses in COVID-19 patients. Cell Host Microbe https://doi.org/10.1016/j.chom.2020.07.005 (2020).

### Items

1. A composition comprising an enriched integrin α10^{high} population of Mesenchymal Stem Cells (MSC), for use in the treatment of one or more disease(s) or trauma(s) of the respiratory system, and/or in connection with transplantation of one or more organs or tissue of the respiratory tract of a mammal.
2. The composition according to item 1, wherein the diseases of the respiratory system is a respiratory disease principally affecting the lung interstitium.
3. The composition for use according to any one of the preceding item, wherein the disease of the respiratory system is acute respiratory distress syndrome (ARDS) and associated disorders.
4. The composition for use according to any one of the preceding items, wherein the disease of the respiratory system is ARDS.
5. The composition for use according to any one of the preceding items, wherein the disease of the respiratory system is Cytokine release syndrome (CRS).
6. The composition for use according to any one of the preceding items, wherein the disease of the respiratory system is cytokine storm syndrome (CSS).
7. The composition for use according to any one of the preceding items, wherein the disease of the respiratory system is cytokine mediated ARDS.
8. The composition for use according to any one of the preceding items, wherein the disease of the respiratory system is ARDS/Respiratory distress syndrome of newborn.
9. The composition for use according to any one of the preceding items, wherein the disease of the respiratory system is ARDS resulting from trauma.
10. The composition for use according to any one of the preceding items, wherein the disease of the respiratory system is ARDS caused by viral or bacterial infection.
11. The composition for use according to any one of the preceding items, wherein the disease of the respiratory system is ARDS resulting from severe acute respiratory syndrome coronavirus 2 (SARS-CoV-2)/COVID-19.
12. The composition for use according to any one of the preceding items, wherein the disease of the respiratory system is ARDS resulting from any other cause.
13. The composition for use according to any one of the preceding items, wherein at least 60% of the cells of the population of MSCs express integrin α10 subunit.
14. The composition for use according to any one of the preceding items, wherein at least 50%, such as at least 55%, such as at least 60%, such as at least 65%, such as at least 70%, such as at least 75%, such as at least 80%, such as at least 85%, such as at least 90%, such as at least 95%, such as at least 96%, such as at least 97%, such as at least 98%, such as at least 99%, such as at least 100% of the total cells comprised in the enriched population population of Mesenchymal Stem Cells (MSC) express integrin α10 subunit.
15. The composition for use according to any one of the preceding items, wherein the MSCs are MHCII negative and/or CD45 negative.
16. The composition for use according to any one of the preceding items, wherein the MSCs express CD44, CD90 and CD105.
17. The composition for use according to any one of the preceding items, wherein the MSC is selected from the group consisting of a mesenchymal stem cells, mesenchymal progenitor cells, and mesenchymal stromal cells; or a mixture thereof.
18. The composition for use according to any one of the preceding items, wherein the cells are induced to express integrin α10 subunit.
19. The composition for use according to any one of the preceding items, wherein the cells are cultured in a culture media comprising mammalian serum and FGF-2.
20. The composition for use according to any one of the preceding items, wherein wherein the cells are cultured in a culture media comprising platelet lysate and/or platelet lysate components.
21. The composition for use according to any one of the preceding items, wherein the cells are cultured in a culture media comprising FGF-2 and platelet lysate and/or platelet lysate components.
22. The composition for use according to any one of the preceding items, wherein the cells are cultured in a culture media comprising mammalian serum and platelet lysate and/or platelet lysate components.
23. The composition for use according to any one of the preceding items, wherein the cells are cultured in a culture media comprising TGFβ.
24. The composition for use according to any one of the preceding items, wherein the cells are cultured in a serum-free culture media comprising platelet lysate and/or platelet lysate components.
25. The composition for use according to any one of the preceding items, wherein the cells are cultured in a serum-free culture media comprising growth factors.
26. The composition for use according to any one of the preceding items, wherein the cells are cultured in a serum-free culture media comprising the growth factors FGF2 and/or TGFβ.
27. The composition for use according to any one of the preceding items, wherein the MSCs are allogeneic or autologous.
28. The composition for use according to any one of the preceding items, wherein the MSCs are administered into the lung or airways.
29. The composition for use according to any one of the preceding items, wherein the population of MSCs is administered via injection.
30. The composition for use according to any one of the preceding items, wherein the population of MSCs is administered in a cell suspension with a pharmaceutically acceptable excipient.
31. The composition for use according to any one of the preceding items, wherein the population of MSCs is formulated into a cell aggregate prior to administration.
32. The composition for use according to any one of the preceding items, wherein the population of MSCs is administered during surgery to repair a damaged lung.
33. The composition for use according to any one of the preceding items, wherein the population of MSCs is administered in connection with lung transplantation.
34. The composition for use according to any one of the preceding items, wherein the mammal is a human, horse, pony, ox, donkey, mule, camelid, cat, dog, pig, or cow.
35. The composition for use according to any one of the preceding items, wherein the mammal is a human.
36. The composition for use according to any one of the preceding items, wherein the MSCs and mammal are from the same species.
37. The composition for use according to any one of the preceding items, wherein the MSCs and mammal are from different species.
38. The composition for use according to any one of the preceding items, wherein the MSCs are derived from adipose tissue, bone marrow, synovial membrane, peripheral blood, cord blood, umbilical cord blood, Wharton's jelly, and/or amniotic fluid.
39. The composition for use according to any one of the preceding items, wherein the MSCs are derived from adipose tissue.
40. The use or the method according to any one of items 76 to 85, wherein the MSCs are derived from bone marrow.
41. The composition for use according to any one of the preceding items, wherein the cells are derived from fetal, neonatal, juvenile or adult MSCs and/or progenitor cells.
42. The composition for use according to any one of the preceding items, wherein the cells are not derived from embryonic cells or from an embryo.
43. The composition for use according to any one of the preceding items, wherein the population of cells is an in vitro cell culture.
44. The composition for use according to any one of the preceding items, wherein the enrichment has been conducted with an anti-integrin α10 antibody.
45. The composition for use according to any one of the preceding items, further comprising an anti-inflammatory agent.

1. A composition comprising integrin alpha10-selected Mesenchymal Stem Cells (MSCs), for use in the treatment of a disease, disorder or trauma of the respiratory system, and/or in connection with transplantation of an organ or tissue of the respiratory tract of a mammal.

2. The composition for use according to claim 1, wherein the disease of the respiratory system is a lower respiratory tract disease.

3. The composition for use according to claim 1, wherein the disease of the respiratory system is a respiratory disease principally affecting the lung interstitium.

4. The composition for use according to claim 1, wherein the disease of the respiratory system is a respiratory disease affecting the airways.

5. The composition for use according to any one of the preceding claims, wherein the disease of the respiratory system is a disease principally affecting the lung interstitium selected from the group consisting of acute respiratory distress syndrome (ARDS), pulmonary oedema , pulmonary eosinophilia, idiopathic interstitial pneumonitis, primary interstitial lung diseases specific to infancy or childhood, interstitial lung diseases associated with systemic diseases, pulmonary alveolar microlithiasis, lymphangioleiomyomatosis, and lipoid pneumonitis.

6. The composition for use according to any one of the preceding claim, wherein the disease of the respiratory system is acute respiratory distress syndrome (ARDS) and/or associated disorders.

7. The composition for use according to any one of the preceding claims, wherein the disease of the respiratory system is ARDS.

8. The composition for use according to any one of the preceding claims, wherein the disease of the respiratory system is cytokine release syndrome (CRS).

9. The composition for use according to any one of the preceding claims, wherein the disease of the respiratory system is cytokine storm syndrome (CSS).

10. The composition for use according to any one of the preceding claims, wherein the ARDS associated disease is multisystem inflammatory syndrome associated with COVID-19.

11. The composition for use according to any one of the preceding claims, wherein the disease of the respiratory system is cytokine mediated ARDS.

12. The composition for use according to any one of the preceding claims, wherein the disease of the respiratory system is ARDS/Respiratory distress syndrome of newborn.

13. The composition for use according to any one of the preceding claim, wherein the disease of the respiratory system is respiratory distress of newborn, such as respiratory distress syndrome of newborn.

14. The composition for use according to any one of the preceding claims, wherein the disease of the respiratory system is ARDS resulting from trauma.

15. The composition for use according to any one of the preceding claims, wherein the disease of the respiratory system is ARDS caused by viral or bacterial infection.

16. The composition for use according to any one of the preceding claims, wherein the disease of the respiratory system is ARDS resulting from severe acute respiratory syndrome coronavirus 2 (SARS-CoV-2)/COVID-19.

17. The composition for use according to any one of the preceding claims, wherein the disease of the respiratory system is ARDS resulting from any other cause.

18. The composition for use according to any one of the preceding claims, wherein at least 60% of the MSCs express integrin α10 subunit.

19. The composition for use according to any one of the preceding claims, wherein at least 50%, such as at least 55%, such as at least 60%, such as at least 65%, such as at least 70%, such as at least 75%, such as at least 80%, such as at least 85%, such as at least 90%, such as at least 95%, such as at least 96%, such as at least 97%, such as at least 98%, such as at least 99%, such as at least 100% of the MSCs express integrin α10 subunit.

20. The composition for use according to any one of the preceding claims, wherein the MSCs are MHC class II, CD45, CD34, CD11b and/or CD19 negative.

21. The composition for use according to any one of the preceding claims, wherein the MSCs express CD73, CD90 and/or CD105.

22. The composition for use according to any one of the preceding claims, wherein the MSCs are selected from the group consisting of a mesenchymal stem cells, mesenchymal progenitor cells, and mesenchymal stromal cells; or a mixture thereof.

23. The composition for use according to any one of the preceding claims, wherein the MSCs are induced to express integrin α10 subunit.

24. The composition for use according to any one of the preceding claims, wherein the MSCs are cultured in a culture media comprising mammalian serum and FGF-2.

25. The composition for use according to any one of the preceding claims, wherein the MSCs are cultured in a culture media comprising platelet lysate and/or platelet lysate components.

26. The composition for use according to any one of the preceding claims, wherein the MSCs are cultured in a culture media comprising FGF-2 and platelet lysate and/or platelet lysate components.

27. The composition for use according to any one of the preceding claims, wherein 3the MSCs are cultured in a culture media comprising mammalian serum and platelet lysate and/or platelet lysate components.

28. The composition for use according to any one of the preceding claims, wherein the MSCs are cultured in a culture media comprising TGFβ.

29. The composition for use according to any one of the preceding claims, wherein the MSCs are cultured in a culture media comprising FGF2.

30. The composition for use according to any one of the preceding claims, wherein the MSCs are cultured in a serum-free culture media comprising platelet lysate and/or platelet lysate components.

31. The composition for use according to any one of the preceding claims, wherein the MSCs are cultured in a serum-free culture media comprising growth factors.

32. The composition for use according to any one of the preceding claims, wherein the MSCs are cultured in a serum-free culture media comprising the growth factors FGF2 and/or TGFβ.

33. The composition for use according to any one of the preceding claims, wherein the MSCs are allogeneic or autologous.

34. The composition for use according to any one of the preceding claims, wherein the composition comprising the MSCs is administered into the lung or airways.

35. The composition for use according to any one of the preceding claims, wherein the composition comprising the MSCs is administered via injection.

36. The composition for use according to any one of the preceding claims, wherein the composition comprising the MSCs is administered parenterally.

37. The composition for use according to any one of the preceding claims, wherein the composition comprising the MSCs is administered via intravenous injection, intramuscular injection and/or intratracheal injection, or any combination thereof.

38. The composition for use according to any one of the preceding claims, wherein the composition comprising the MSCs is administered in a cell suspension with a pharmaceutically acceptable excipient.

39. The composition for use according to any one of the preceding claims, wherein the composition comprising the MSCs is administered during surgery to repair a damaged lung.

40. The composition for use according to any one of the preceding claims, wherein the composition comprising the MSCs is administered in connection with lung transplantation.

41. The composition for use according to any one of the preceding claims, wherein the mammal is a human.

42. The composition for use according to any one of the preceding claims, wherein the MSCs and mammal are from the same species.

43. The composition for use according to any one of the preceding claims, wherein the MSCs and mammal are from different species.

44. The composition for use according to any one of the preceding claims, wherein the MSCs are derived from adipose tissue, bone marrow, synovial membrane, peripheral blood, cord blood, umbilical cord blood, Wharton's jelly, and/or amniotic fluid.

45. The composition for use according to any one of the preceding claims, wherein the MSCs are derived from adipose tissue.

46. The composition for use according to any one of the preceding claims, wherein the MSCs are derived from bone marrow.

47. The composition for use according to any one of the preceding claims, wherein the MSCs are derived from fetal, neonatal, juvenile or adult MSCs and/or progenitor cells.

48. The composition for use according to any one of the preceding claims, wherein the MSCs are not derived from embryonic cells or from an embryo.

49. The composition for use according to any one of the preceding claims, wherein the MSCs are an in vitro cell culture.

50. The composition for use according to any one of the preceding claims, wherein the selection of MSCs has been conducted with an anti-integrin α10 antibody.

51. The composition for use according to any one of the preceding claims, further comprising an anti-inflammatory and/or immunomodulatory agent.

52. Use of a composition comprising integrin α10-selected Mesenchymal Stem Cells (MSCs), for the manufacture of a medicament for the treatment of a disease, disorder or trauma of the respiratory system, and/or in connection with transplantation of an organ or tissue of the respiratory tract of a mammal.

53. A method of treatment of a disease, disorder or trauma of the respiratory system of a mammal, the method comprising administering a therapeutically effective amount of a composition comprising integrin α10-selected Mesenchymal Stem Cells (MSCs).

54. A method of treatment or promotion of a transplantation of an organ or tissue of the respiratory tract of a mammal, the method comprising administering a therapeutically effective amount of a composition comprising integrin α10-selected Mesenchymal Stem Cells (MSCs).

55. A method of preventing blood clotting in a mammal in connection with a disease, disorder, or trauma of the respiratory system, and/or in connection with transplantation of an organ or tissue of the respiratory tract of a mammal, the method comprising administering a therapeutically effective amount of a composition comprising integrin α10-selected Mesenchymal Stem Cells (MSCs).

56. A method of promoting hemodynamic stability in connection with a disease, disorder, or trauma of the respiratory system, and/or in connection with transplantation of an organ or tissue of the respiratory tract of a mammal, the method comprising administering a therapeutically effective amount of a composition comprising integrin α10-selected Mesenchymal Stem Cells (MSCs).

57. A method of reducing the need for inotropic support in connection with a disease, disorder, or trauma of the respiratory system, and/or in connection with transplantation of an organ or tissue of the respiratory tract of a mammal, the method comprising administering a therapeutically effective amount of a composition comprising integrin α10-selected Mesenchymal Stem Cells (MSCs).

58. A method of improving oxygenation capacity in connection with a disease, disorder, or trauma of the respiratory system, and/or in connection with transplantation of an organ or tissue of the respiratory tract of a mammal, the method comprising administering a therapeutically effective amount of a composition comprising integrin α10-selected Mesenchymal Stem Cells (MSCs).

59. A method of preventing tissue damage, for example structural tissue damage, in connection with a disease, disorder, or trauma of the respiratory system, and/or in connection with transplantation of an organ or tissue of the respiratory tract of a mammal, the method comprising administering a therapeutically effective amount of a composition comprising integrin α10-selected Mesenchymal Stem Cells (MSCs).

60. A method of reverting tissue damage, for example structural tissue damage, in connection with a disease, disorder, or trauma of the respiratory system, and/or in connection with transplantation of an organ or tissue of the respiratory tract of a mammal, the method comprising administering a therapeutically effective amount of a composition comprising integrin α10-selected Mesenchymal Stem Cells (MSCs).

61. The method according to any one of claims 59 to 60, wherein the tissue damage is lung tissue damage.

62. The method according to any one of claims 59 to 61, wherein the tissue damage is damage of the interstitial tissue, damage of the alveolar septa, damage of the airways, damage of the vasculature and/or damage of the nervous system.

63. A method of reducing neutrophil counts in connection with a disease, disorder, or trauma in connection with a disease, disorder, or trauma of the respiratory system, and/or in connection with transplantation of an organ or tissue of the respiratory tract of a mammal, the method comprising administering a therapeutically effective amount of a composition comprising integrin α10-selected Mesenchymal Stem Cells (MSCs).

64. A method of increasing lymphocyte counts in connection with a disease, disorder, or trauma in connection with a disease, disorder, or trauma of the respiratory system, and/or in connection with transplantation of an organ or tissue of the respiratory tract of a mammal, the method comprising administering a therapeutically effective amount of a composition comprising integrin α10-selected Mesenchymal Stem Cells (MSCs).

65. A method of decreasing proinflammatory cytokines in connection with a disease, disorder, or trauma in connection with a disease, disorder, or trauma of the respiratory system, and/or in connection with transplantation of an organ or tissue of the respiratory tract of a mammal, the method comprising administering a therapeutically effective amount of a composition comprising integrin α10-selected Mesenchymal Stem Cells (MSCs).

66. The method according to claim 65, wherein the proinflammatory cytokines are selected from the group consisting of interleukin 12 (IL-12), IL-1β, IL-6 and IL-4, or any combination thereof.

67. The method according to any one of claims 65 to 66, wherein the proinflammatory cytokines are decreased in blood and/or bronchoalveolar lavage fluid.

68. A method of increasing interferon-α in connection with a disease, disorder, or trauma in connection with a disease, disorder, or trauma of the respiratory system, and/or in connection with transplantation of an organ or tissue of the respiratory tract of a mammal, the method comprising administering a therapeutically effective amount of a composition comprising integrin α10-selected Mesenchymal Stem Cells (MSCs).

69. A composition comprising integrin alpha10-selected Mesenchymal Stem Cells (MSCs), for use in a method of
preventing blood clotting,
promoting hemodynamic stability,
reducing the need for inotropic support,
improving oxygenation capacity,
preventing tissue damage, for example structural tissue damage,
reverting tissue damage, for example structural tissue damage,
reducing neutrophil counts,
increasing lymphocyte counts,
decreasing proinflammatory cytokines, and/or
increasing interferon-α,
in a mammal in connection with a disease, disorder, or trauma of the respiratory system, and/or in connection with transplantation of an organ or tissue of the respiratory tract of a mammal.

70. The method according to claim 69, wherein the tissue damage is lung tissue damage.

71. The method according to any one of claims 69 to 70, wherein the tissue damage is damage of the interstitial tissue, damage of the alveolar septa, damage of the airways, damage of the vasculature, damage of muscle and/or damage of the nervous system.

72. The method according to claim 69, wherein the proinflammatory cytokines are selected from the group consisting of interleukin 12 (IL-12), IL-1β, IL-6 and IL-4, or any combination thereof.

73. The method according to any one of claims 69 to 72, wherein the proinflammatory cytokines are decreased in blood and/or bronchoalveolar lavage fluid.
